(19) United States
(12) Patent Application Publication
(10) Pub. No.: US 2025/0152027 A1
(43) Pub. Date: May 15, 2025

(54) Circulatory Assist Systems and Methods

(71) Applicant: TC1 LLC, St. Paul, MN (US)

(72) Inventors: Alexander Medvedev, Ann Arbor, MI (US); Shunzhou Yu, Ann Arbor, MI (US); Ren You, Windsor (CA)

(73) Assignee: TC1 LLC, St. Paul, MN (US)

(21) Appl. No.: 19/027,576

(22) Filed: Jan. 17, 2025

Related U.S. Application Data
(63) Continuation of application No. 18/212,844, filed on Jun. 22, 2023, now Pat. No. 12,213,766.
Application No. 18/212,844 is a continuation of application No. 17/083,057, filed on Oct. 28, 2020, now Pat. No. 11,712,167.
Application No. 17/083,057 is a continuation of application No. 16/714,287, filed on Dec. 13, 2019, now Pat. No. 10,856,748.
Application No. 16/714,287 is a continuation of application No. 16/050,889, filed on Jul. 31, 2018, now Pat. No. 10,506,935.
Application No. 16/050,889 is a continuation of application No. 15/041,716, filed on Feb. 11, 2016, now Pat. No. 10,052,420.
(60) Provisional application No. 62/114,886, filed on Feb. 11, 2015.

(51) Int. Cl. A61B 5/024; A61M 1/00; A61M 60/148; A61M 60/178; A61M 60/408; A61M 60/515; A61M 60/538

(52) U.S. Cl. CPC A61B 5/024; A61B 5/02438; A61M 1/00; A61M 60/178; A61M 60/408; A61M 60/515; A61M 60/538; A61M 60/148; A61M 2205/3334; A61M 2230/04

(57) ABSTRACT

Circulatory assist systems and related methods provide for pumping of blood from a ventricle to an artery. A circulatory assist system includes a ventricular assist device, a motor control circuit, a controller, a sensing circuit, and a blood flow cannula. The ventricular assist device includes a housing, a stator assembly, and a blood flow impeller. The blood flow impeller includes a first disk portion, a second disk portion, and vanes extending between the first disk portion and the second disk portion. Each of the first disk portion and the second disk portion includes embedded magnetic segments for rotation and levitation of the blood flow impeller. The first disk portion has central aperture configured for transit of a blood flow received through the blood flow inlet into the blood flow impeller for impelling radially outwardly between the first disk portion and the second disk portion via the vanes.

### CROSS-REFERENCE TO RELATED APPLICATIONS

The present application is a Continuation of U.S. patent application Ser. No. 18/212,844 filed Jun. 22, 2023 (Allowed); which is a Continuation of U.S. Ser. No. 17/083,057 filed Oct. 28, 2020 (now U.S. Pat. No. 11,712,167); which is a Continuation of U.S. Ser. No. 16/714,287 filed Dec. 13, 2019 (now U.S. Pat. No. 10,856,748); which is a Continuation of U.S. Ser. No. 16/050,889 filed Jul. 31, 2018 (now U.S. Pat. No. 10,506,935); which is a Continuation of U.S. Ser. No. 15/041,716, filed Feb. 11, 2016 (now U.S. Pat. No. 10,052,420); which claims priority to U.S. Provisional Appln No. 62/114,886 filed Feb. 11, 2015; the disclosures which are incorporated herein by reference in their entirety for all purposes.

This application is also related to commonly assigned U.S. patent application Ser. No. 13/873,551 filed Apr. 30, 2013 (now U.S. Pat. No. 9,713,663), entitled “CARDIAC PUMP WITH SPEED ADAPTED FOR VENTRICLE UNLOADING.” The disclosure which is incorporated herein by reference in its entirety for all purposes.

### BACKGROUND OF THE INVENTION

A left ventricular assist device (LVAD) and/or other devices may be used to provide long-term support for heart failure patients or patients suffering from other heart related conditions. Traditionally, many such devices assist heart functioning by generating a continuous blood flow using a constant pumping speed set by clinician based on the patient's physiologic conditions at that time when the particular device is implanted.

However, the natural cardiac cycle of a human being (or other animals) does not usually generate a continuous and constant blood flow. Instead, flow is highest during the systole of a cardiac cycle, and then decreased during the diastole of the cardiac cycle. Thus the heart and the implanted device operate in different fashions (i.e., non-constant versus constant flow) which may be detrimental to the patient.

Embodiments of the present invention provide systems and methods for determining characteristics of a cardiac cycle, so that operation of LVAD and/or other devices may be altered in a dynamic manner when used in a human or other animal experiencing heart related conditions.

### BRIEF SUMMARY OF THE INVENTION

In one aspect, a method for synchronizing operation of a heart assist pump device to a patient's cardiac cycle is provided. The method may include obtaining a signal from a motor of a heart assist pump device anPd filtering the signal to remove noise. The method may also include determining a speed synchronization start point at which time the motor of the heart assist pump device will begin a change in speed of operation based on the filtered signal. The method may further include modulating a speed of the motor of the heart assist pump device to a target speed at the speed synchronization start point, thereby synchronizing the change in speed of operation with a patient's cardiac cycle.

In another aspect, a heart assist pump device is provided. The device may include a motor and a controller. The controller may be configured to obtain frequency range data from the motor and to determine a speed synchronization start point at which time the motor of the heart assist pump device will begin a change in speed of operation based on the frequency range data. The controller may also be configured to modulate a speed of the motor to a target speed at the speed synchronization start point, thereby synchronizing the change in speed of operation with a patient's cardiac cycle.

### BRIEF DESCRIPTION OF THE DRAWINGS

The present invention is described in conjunction with the appended figures:

FIG. 1 shows a top level block diagram of a LVAD pump control system of one embodiment of the invention;

FIG. 2 shows one speed modulation architecture of one embodiment of the invention;

FIG. 3 shows one method of identifying a pulse period of a heart beat of one embodiment of the invention;

FIG. 4 motor data converted to heart beat information and various characteristics thereof pertinent to methods and system of the invention;

FIG. 5 is a block diagram of an exemplary computer system capable of being used in at least some portion of the apparatuses or systems of the present invention, or implementing at least some portion of the methods of the present invention; and

FIGS. 6A and 6B show four possible ways to determine an initial speed synchronization start point.

FIG. 7 is a diagram of a circulatory assist system as one example of an implantable pump employing the present invention.

FIG. 8 is an exploded, perspective view of a portion of a centrifugal pump of a type that can be used in the present invention.

FIG. 9 is a diagram showing a prior art variable speed profile synchronized with a cardiac cycle.

FIG. 10 is a diagram showing a variable speed profile of the present invention.

FIG. 11 is a diagram showing a variable speed profile in greater detail.

FIG. 12 is a block diagram showing a pump motor and control system of the present invention.

FIG. 13 is a diagram showing pump load and speed according to an embodiment using a constant current mode.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth herein.

Specific details are given in the following description to provide a thorough understanding of the embodiments. However, it will be understood by one of ordinary skill in the art that the embodiments may be practiced without these specific details. For example, with regard to any specific embodiment discussed herein, any one or more details may or may not be present in all versions of that embodiment. Likewise, any detail from one embodiment may or may not be present in any particular version of another embodiment discussed herein. Additionally, well-known circuits, systems, processes, algorithms, structures, and techniques may be shown without unnecessary detail in order to avoid obscuring the embodiments. The absence of discussion of any particular element with regard to any embodiment herein shall be construed to be an implicit contemplation by the disclosure of the absence of that element in any particular version of that or any other embodiment discussed herein.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

The term “machine-readable medium” includes, but is not limited to portable or fixed storage devices, optical storage devices, wireless channels and various other mediums capable of storing, containing or carrying instructions and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments of the invention may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. One or more processors may perform the necessary tasks.

In some embodiments, a left ventricular assist device (LVAD) or other device may be intended to provide the long-term support for a heart failure patient or a patient suffering from another condition. Many such devices generate a continuous blood flow using a constant pumping speed set by clinician or other process based on the patient's physiologic conditions at that time when such device is implanted. However, there is the potential to vary the speed of the device to be synchronized to the natural cardiac cycle by modulating the speed based on the natural cardiac cycle. Using this approach, the pump speed is increased during systole of a cardiac cycle (the time of highest flow) and decreased during diastole (the time of lowest flow), so that a maximum unloading of a weakened ventricle may be obtained. This may establish stable hemodynamic conditions and enables a variation of the aortic pulse pressure, while keeping the organ perfusion at an even level to benefit the patient's recovery. Although the heart is weakened, it is still beating. The LVAD may support the beating heart such that when the heart pumps the resistance met by the pump goes down and vice versa. This would be seen as a change in the back emf and current. In some embodiments, the change in current may depend on the control scheme. For example, the LVAD may be designed to maintain a set motor speed (rpm). The current needed to maintain the speed goes down during pumping (systole). In other embodiments, the LVAD may be designed to maintain a set flow rate, causing the current to go down during systole. It will be appreciated that the LVAD could be designed to just apply a set current, in which case it doesn't matter what the heart is doing. The flow rate will then go up when the pump and heart are pushing fluid at the same time.

In some embodiments the pump speed of a LVAD or other device may be precisely synchronized to the systolic phases of the cardiac cycle in a reliable real-time mode regardless of the irregular heart beats. This may prevent a lack of synchrony which may cause ventricular load fluctuation or even overloading of the heart which can increase the occurrence of adverse events and affect the recovery of the patient. Unsynchronized increases in pump speed could also increase the risk of ventricular suction, particularly at the end of systole when the ventricle could be nearly empty. Embodiments of the invention reduce such risks by properly identifying regular heart beats and the proper time to increase pump speed relative thereto.

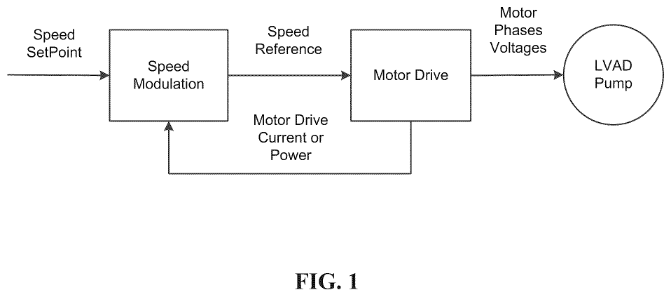

Embodiments of the invention implement real-time speed modulation to at least more precisely synchronize LVAD pumps or other devices with the heart beat cycle that allow for increasing the pump speed before the systolic phase and reducing the speed before the end of systole. FIG. 1 shows a top level block diagram of a LVAD pump control system (or control system for other device) with speed modulation. In this control system, motor drive current or power signal is used as the input of the speed modulation since it reflects the heart beat cycle pattern. By extracting the motor drive current or power signal features, speed synchronization time points within the heart beat cycles can be determined. Based on the speed synchronization time points, the speed set-point configured by clinician or other method can be modulated to the targeted speed reference automatically at the right time for a motor drive of a pump or other device to achieve the required synchronicity.

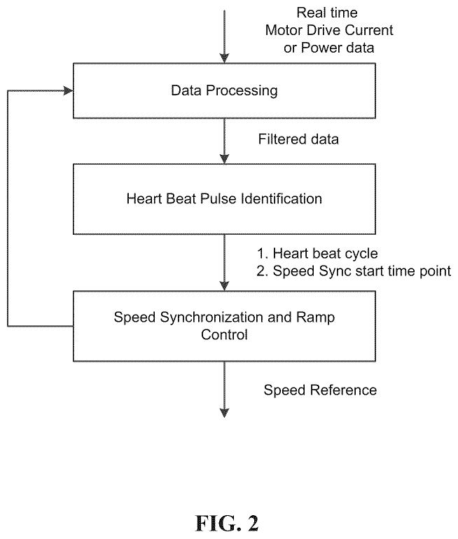

The architecture of speed modulation is shown in FIG. 2 which consists of three main stages:
Stage 1—Data Processing—A filter is employed to obtain reasonable frequency range data from the raw motor current or power signal data which is concurrent with, and representative of, the heart beat cycle.
Stage 2—Heart Beat Pulse Identification—The heart beat cycle features are identified from the filtered motor current or power data from Stage 1 to determines the speed synchronization start time point (i.e., the point in time in which the pump speed should increase).
Stage 3—Speed Synchronization and Ramp Control—Based on the speed synchronization start point identified in Stage 2, a pump motor is controlled to synchronize speed increases thereof with heart beats. A targeted speed reference for the motor drive, with ramp up and down control, is specified by a clinician or other method.

At Stage 1, high frequency noise data which is out of the general heart beat range (i.e. less than 5 Hz (300 beats/min)) is filtered out of the motor current or power data. Any kind of digital filter, for example, an infinite impulse response filter (IIR) or finite impulse response filter (FIR), may be employed, but the phase delay and computational load may need to be considered when implemented it into an embedded LVAD controller. In one embodiment, a second order IIR is employed.

At Stage 2, the pulse period of heart beat is identified from the filtered motor current or power data from Stage 1. In some embodiments, at least two consecutive and complete prior-occurring heart beats may be analyzed to anticipate the current heart beat cycle features. In other embodiments, the two complete prior-occurring heart beats may not be consecutive, or more than two complete prior-occurring heart beats may be analyzed, either consecutive or non-consecutive. In some embodiments, more than two complete prior-occurring heart beats may be analyzed. For example, three, four, five, or any specific number of heart beats greater than five may be analyzed depending on the embodiment. However, using more than the last two consecutive and complete prior-occurring heart beats may involve older heart beat history data which may include irregular heart beats or inconsistent data, thereby reducing the accuracy of the predicted current heart beats cycle features.

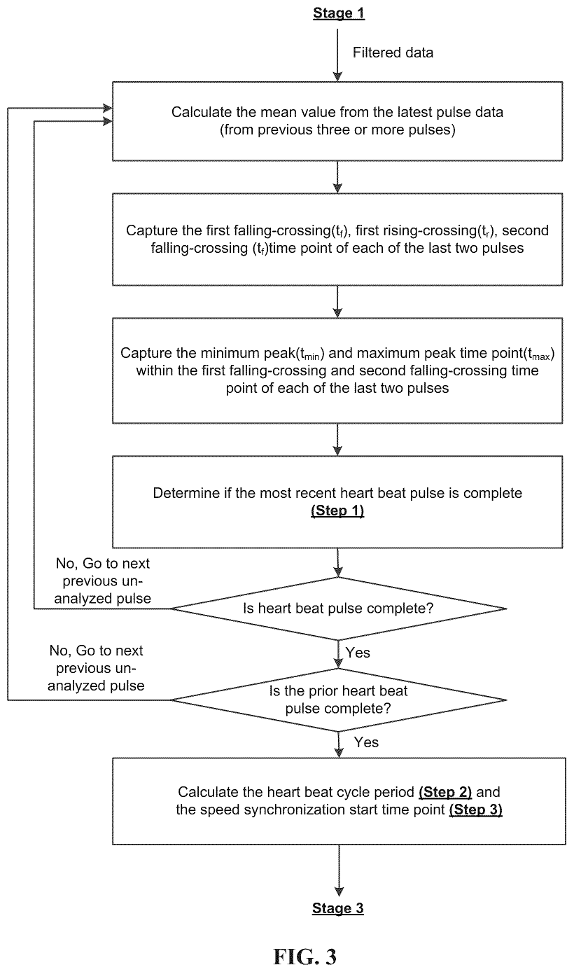

Stage 2 involves three separate steps as shown in FIG. 3:

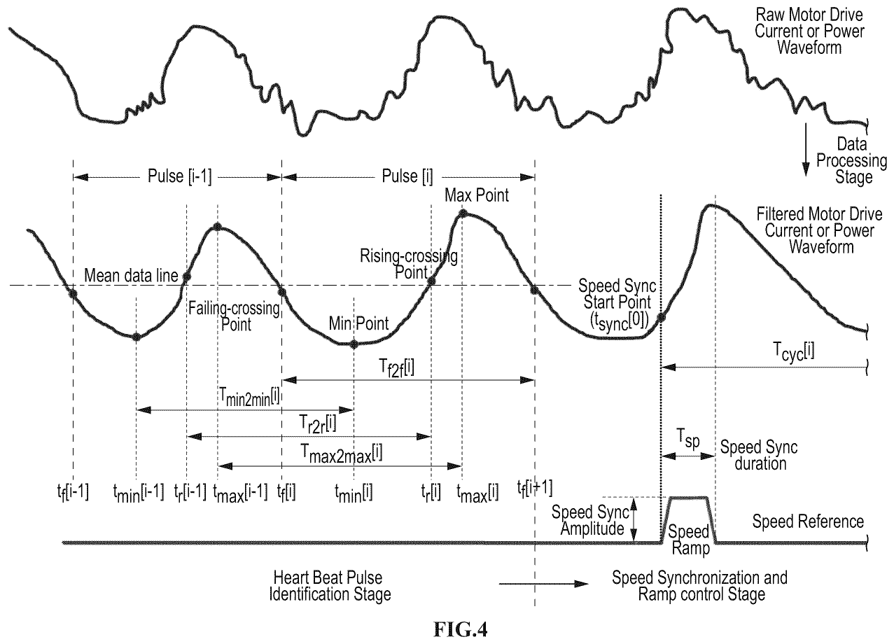

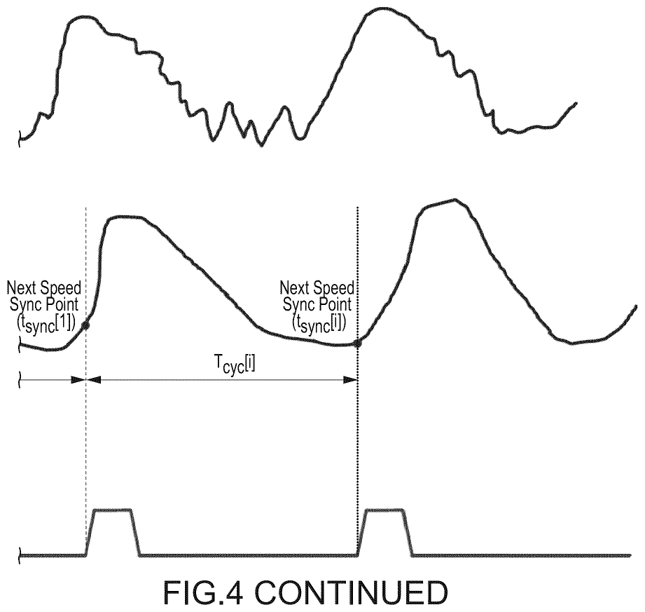

Step 1—Determine if each pulse is complete—To determine if a pulse is complete, characteristics of the pulse may first be determined from the data provided from Stage 1. Those characteristics may include the following (see FIG. 4 for reference):
The mean amplitude value from the previous three or more pulses
The maximum amplitude value from the pulse to be analyzed
The minimum amplitude value from the pulse to be analyzed
The first falling-crossing time (t_{f}) which is the point at which the pulse first crosses the mean value on the downslope
The first rising-crossing time (t_{f}) which is the point at which the pulse crosses the mean value on the upslope
The second falling-crossing time (t_{f}) which is the point at which the pulse crosses the mean value on the downslope for a second time
The minimum peak time point (tₘᵢₙ) for the minimum amplitude value
The maximum peak time point (tₘₐₓ) for the maximum amplitude value

The following rules are then used to determine if two consecutive prior pulses are complete pulses. Both rules must be satisfied to allow the two pulses to be used as a reference for Step 2 of the process which determines the heart beat cycle period.

Rule 1: The amplitude difference between the maximum amplitude and the mean amplitude must satisfy the following:

$${c}_{1} {Diff}_{max 2 Min} [ i ] < {Diff}_{max 2 Mean} [ i ] < {c}_{2} {Diff}_{max 2 Min} [ i ]$$

Where,
Diff_{max2Mean}[i]=the difference between maximum amplitude and mean data at pulse[i];
Diff_{max2Min}[i]=the difference between maximum and minimum amplitude at pulse[i]; and
c₁, c₂ are two constant coefficients.

In one embodiment c₁=0.375 and c₂=0.75, though in other embodiments other values of c₁ and c₂ may be possible.

Rule 2: The four pulse periods from different time points must satisfy the following:

$${T}_{cyc \_ min} < \{ {T}_{f 2 f} [ i ] , {T}_{r 2 r} [ i ] , {T}_{min 2 min} [ i ] , {T}_{max 2 max} [ i ] \} < {T}_{cyc \_ max}$$

Where,
T_{f2f}[i]=A pulse period from the last falling-crossing time point to current falling-crossing time point;
T_{f2f}[i]=A pulse period from the last rising-crossing time point to current rising-crossing time point;
Tₘᵢₙ₂ₘᵢₙ[i]=A pulse period from the last minimum time point to current minimum time point;
Tₘₐₓ₂ₘₐₓ[i]=A pulse period from the last maximum time point to current maximum time point; and
T_{cyc_min}, T_{cyc_max} are the limitations of a pulse period of heart beat.

In one embodiment T_{cyc_min}=0.3 seconds (200 beats/min), T_{cyc_max}=1.25 seconds (48 beats/min), though in other embodiments other values of T_{cyc_min} and T_{cyc_max} may be possible.

If these rules are not satisfied for two prior consecutive pulses, then such pulses are not adjudged to be complete pulses and pulses prior to the non-complete pulses are then analyzed until two prior consecutive complete pulse are located. Step 2 is then commenced based on such pulses.

Step 2—Determine the pulse period—To determine the pulse period the median value of the previously discussed four pulse periods is determined per the below:

$${T}_{cyc} [ i ] = Median \{ {T}_{f 2 f} [ i ] , {T}_{r 2 r} [ i ] , {T}_{min 2 min} [ i ] , {T}_{max 2 max} [ i ] \}$$

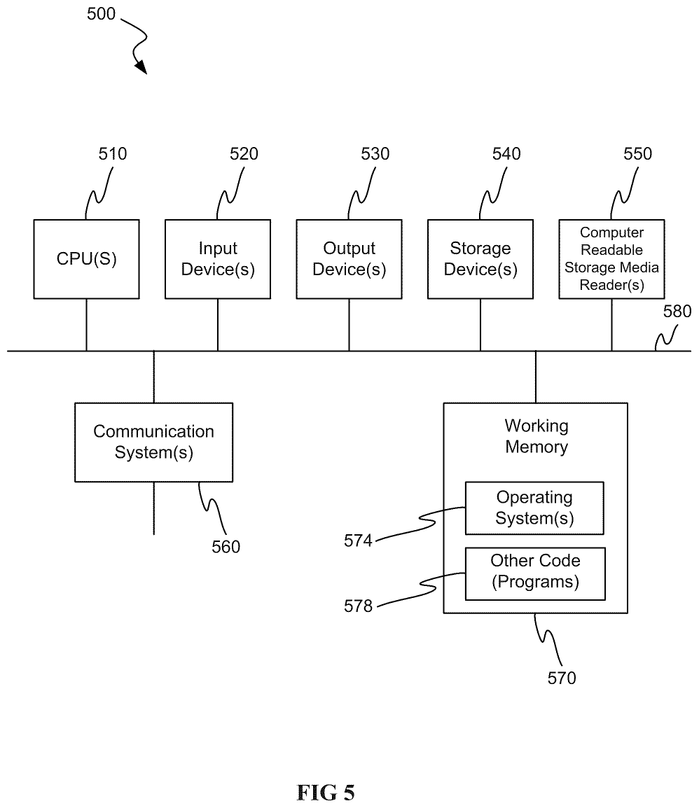

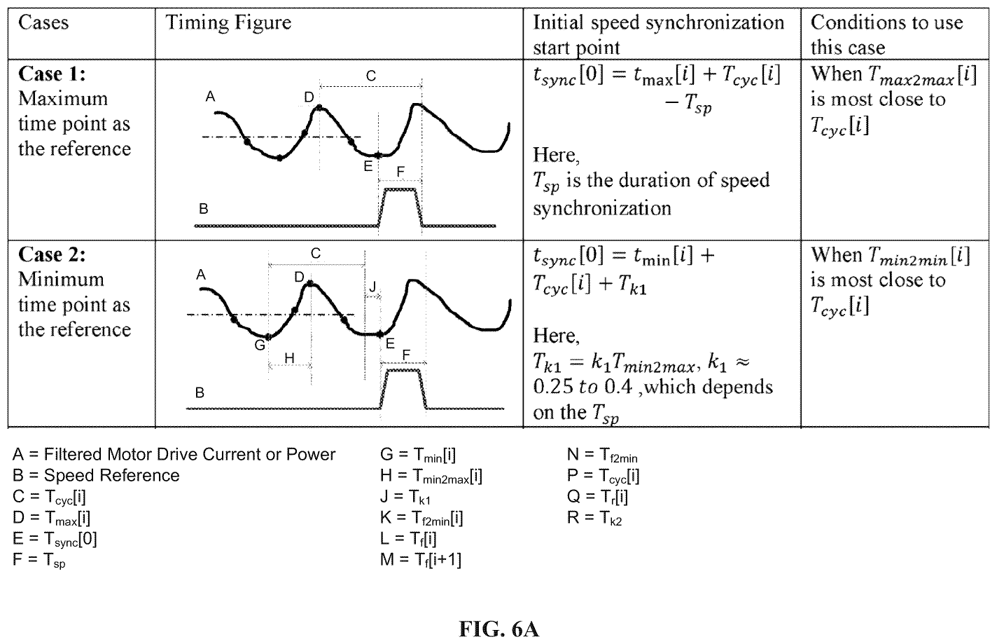

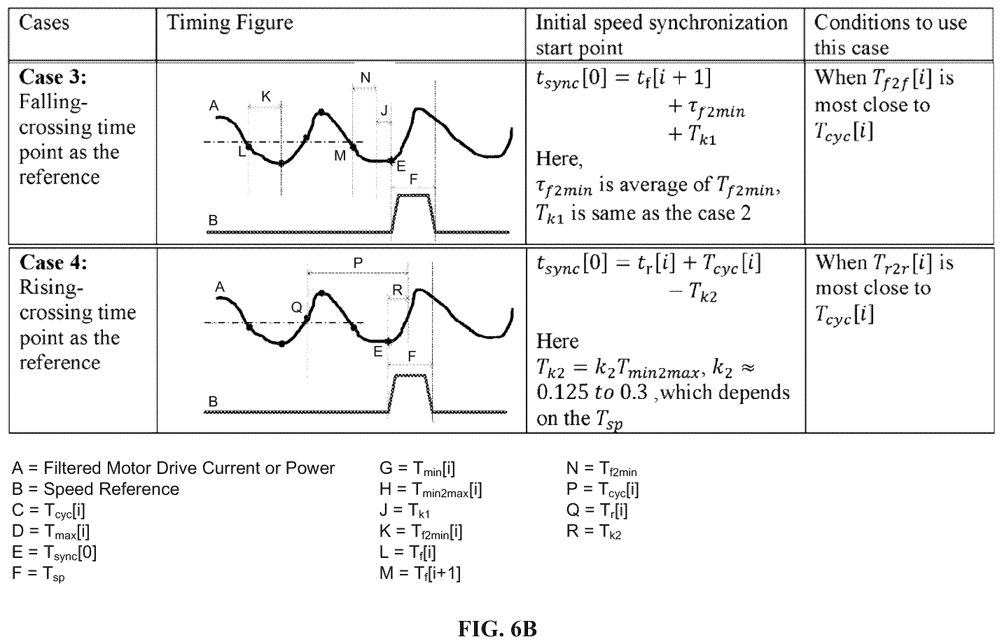

Step 3—Calculate initial speed synchronization start point—There are at least four possible ways to determine an initial speed synchronization start point (t_{sync}[0]) as shown in the tables in FIGS. 6A and 6B. In the case where two conditions of different cases in the tables are indicated as being equally applicable (for example, under Case 1 and Case 2, the difference between Tₘₐₓ₂ₘₐₓ[i] and T_{cyc}[i] is equal to the difference between Tₘᵢₙ₂ₘᵢₙ[i] and T_{cyc}[i]), the lower number Case has priority and is employed (in the example, Case 1, instead of Case 2).

After completion of Step 2 and Step 3, the process continues to Stage 3, where based on the speed synchronization start point identified in Stage 2, a pump motor is controlled to synchronize speed increases thereof with heart beats. Considering all the timing offsets such as the data filter timing delay, the phase shift between left ventricle pressure and pumping flow and motor drive current or power, pump speed ramp up and down time, all the next series of speed synchronization time points can be finalized as:

$${t}_{sync} [ j ] = {t}_{sync} [ 0 ] - {T}_{offset} + ( j - 1 ) * {T}_{cyc} [ i ]$$

Where T_{offset}<Tₘᵢₙ₂ₘₐₓ and in one possible embodiment T_{offset}≈T_{offset}≈40˜80 ms, and J is equal to the sequential heart beat to be synchronized (i.e., J=1 at the first heart beat, J=2 at the second heart beat, etc.).

This synchronized heart beat count (J) should not be too large, since the speed synchronization at one round may rely on the results of Stages 1 and 2, which may not be matched with the current heart beat features at after some while probably due to the patient's physiology or other factors, and thus possibly cause asynchrony between the pump and heartbeat. Therefore, to get the precise real-time synchronization, it may be necessary to identify the latest heart beat cycle features and start the synchronization again after several synchronized heart beat counts. Thus Jₘₐₓ may equal 10, 9, 8, 7, or fewer beats in some embodiments, though in other embodiments may exceed 10, prior to Stages 1-3 being re-initiated to ensure asynchrony between the pump and heartbeat does not occur.

FIG. 5 is a block diagram illustrating an exemplary processing or computer system 500 in which embodiments of the present invention may be implemented. This example illustrates a processing or computer system 500 such as may be used, in whole, in part, or with various modifications, to provide the functions of a processor controlling and receiving data back from an LVAD or other device, and/or other components of the invention such as those discussed above. For example, various functions of such processor may be controlled by the computer system 500, including, merely by way of example, receiving current or power data back from the pump motor, data processing with regard to previous heart pulses, and speed synchronization of the pump based on such data, etc.

The computer system 500 is shown comprising hardware elements that may be electrically coupled via a bus 590. The hardware elements may include one or more central processing units 510, one or more input devices 520 (e.g., data acquisition subsystems), and one or more output devices 530 (e.g., control subsystems). The computer system 500 may also include one or more storage device 540. By way of example, storage device(s) 540 may be solid-state storage device such as a random access memory (“RAM”) and/or a read-only memory (“ROM”), which can be programmable, flash-updateable and/or the like.

The computer system 500 may additionally include a computer-readable storage media reader 550, a communications system 560 (e.g., a network device (wireless or wired), a Bluetooth™ device, cellular communication device, etc.), and working memory 580, which may include RAM and ROM devices as described above. In some embodiments, the computer system 500 may also include a processing acceleration unit 570, which can include a digital signal processor, a special-purpose processor and/or the like.

The computer-readable storage media reader 550 can further be connected to a computer-readable storage medium, together (and, optionally, in combination with storage device(s) 540) comprehensively representing remote, local, fixed, and/or removable storage devices plus storage media for temporarily and/or more permanently containing computer-readable information. The communications system 560 may permit data to be exchanged with a network, system, computer and/or other component described above.

The computer system 500 may also comprise software elements, shown as being currently located within a working memory 580, including an operating system 584 and/or other code 588. It should be appreciated that alternate embodiments of a computer system 500 may have numerous variations from that described above. For example, customized hardware might also be used and/or particular elements might be implemented in hardware, software (including portable software, such as applets), or both. Furthermore, connection to other computing devices such as network input/output and data acquisition devices may also occur.

Software of computer system 500 may include code 588 for implementing any or all of the function of the various elements of the architecture as described herein. Methods implementable by software on some of these components have been discussed above in more detail.

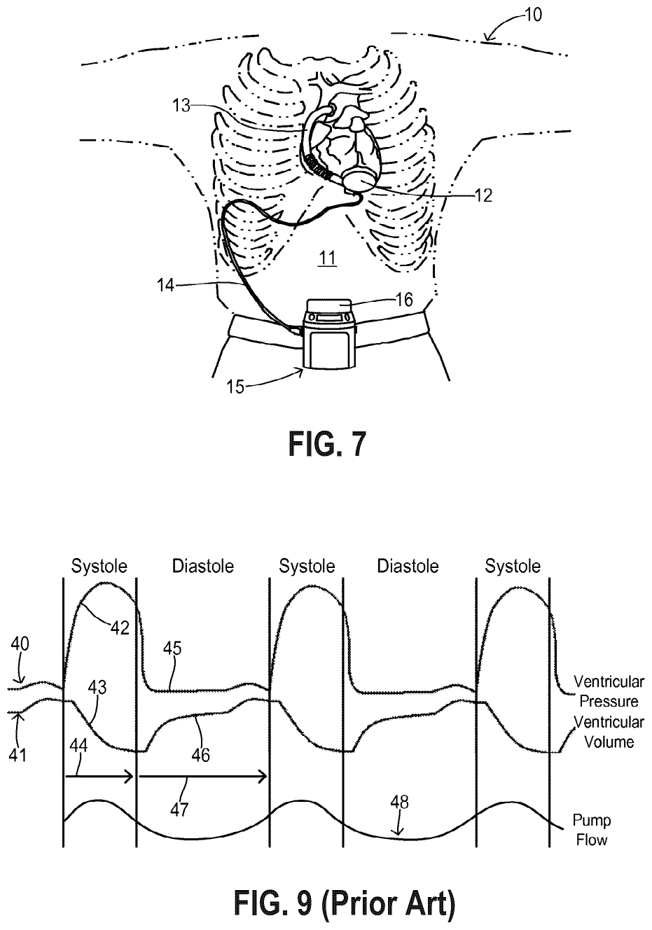

Referring to FIG. 7, a patient 10 is shown in fragmentary front elevational view. Surgically implanted either into the patient's abdominal cavity or pericardium 11 is the pumping unit 12 of a ventricular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power cable 14 extends from pumping unit 12 outwardly of the patient's body via an incision to a compact control unit 15 worn by patient 10. Control unit 15 is powered by a main battery pack 16 and/or an external AC power supply and an internal backup battery. Control unit 15 includes a commutator circuit for driving a motor within pumping unit 12.

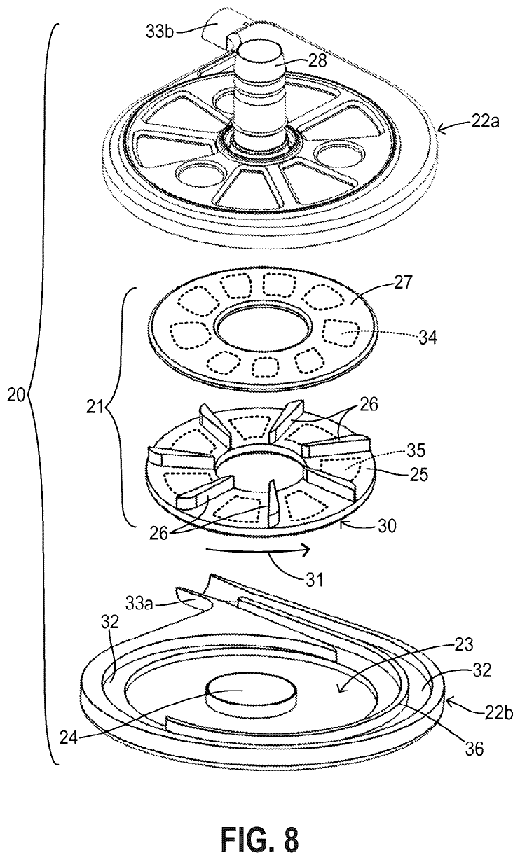

FIG. 8 shows a centrifugal pump unit 20 having an impeller 21 and a pump housing having upper and lower halves 22a and 22b. Impeller 21 is disposed within a pumping chamber 23 over a hub 24. Impeller 21 includes a first plate or disc 25 and a second plate or disc 27 sandwiched over a plurality of vanes 26. Second disc 27 includes a plurality of embedded magnet segments 34 for interacting with a levitating magnetic field created by a levitation magnet structure (not shown) that would be disposed against or incorporated in housing 22a. First disc 25 also contains embedded magnet segments 35 for magnetically coupling with a magnetic field from a motor (not shown) disposed against or incorporated in housing 22b. Housing 22a includes an inlet 28 for receiving blood from a patient's ventricle and distributing it to vanes 26. Impeller 21 is preferably circular and has an outer circumferential edge 30. By rotatably driving impeller 21 in a pumping direction 31, the blood received at an inner edge of impeller 21 is carried to outer circumferential 30 and enters a volute region 32 within pumping chamber 23 at an increased pressure. The pressurized blood flows out from an outlet 33 formed by housing features 33a and 33b. A flow-dividing guide wall 36 may be provided within volute region 32 to help stabilize the overall flow and the forces acting on impeller 21.

FIG. 9 shows a ventricular pressure curve 40 and a ventricular volume curve 41 according to a typical cardiac cycle. A pressure pulse 42 and a volume ejection 43 correspond with a systolic phase 44. A low ventricular pressure 45 and increasing ventricular volume 46 correspond with a diastolic phase 47. The start of systole corresponds with the time that the mitral or tricuspid valve closes, and the start of diastole corresponds with the time that the aortic valve or pulmonary valve closes. Curve 48 shows a pulsatile pump flow in which the pump speed is synchronously varied in order to provide an increased speed during systolic phase 44 and a decreased speed during diastolic phase 47. As explained above, this conventional pulsatile flow does not significantly unload the ventricle at the beginning of systole.

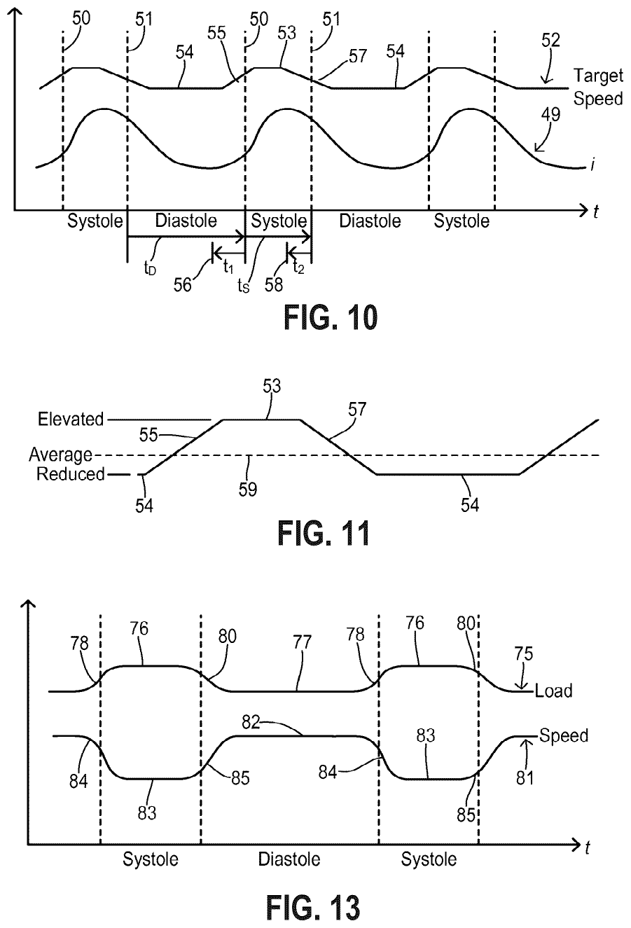

FIG. 10 and FIG. 11 show a first embodiment of the invention for providing a variable speed mode with certain speed changes preceding the beginning of systole 50 and the beginning of diastole 51. An impeller speed curve 52 provides a pulsatile flow between an elevated speed 53 and a reduced speed 54. Curve 49 shows a representative current vector applied to the motor by the motor controller in order to generate a corresponding target speed. Thus, an increase in current generates a speed increase and a decrease in current generates a speed decrease. The target speed includes a ramping up at segment 55 from reduced speed 54 to elevated speed 53, wherein the ramping up begins during diastole. The time of increase and the slope of the increase are configured to provide an increasing flow before systole begins to provide a reduced ventricular pressure that allows a weak ventricle to contract with reduced resistance. The controlled motor current 49 begins to increase at a time 56 which precedes systole by a time period t₁. The beginning of ramping up segment 55 of the speed may lag the current increase but still occurs prior to the beginning of systole 50. Preferably, time 56 is scheduled by the motor controller at time t₁ before the next expected occurrence of systole 50 such that the ramping up begins at a moment between about 50% to about 90% into the diastolic phase. Thus, denoting the length of the diastolic phase as t_{D}, the ratio t₁/t_{D} is preferably between 0.1 and 0.5.

To help avoid collapse of the ventricle toward the end of systole or during diastole, impeller speed 52 preferably ramps down at segment 57 from elevated speed 53 to reduced speed 54. Segment 57 begins during the systolic phase of the cardiac cycle (i.e., before the beginning of diastole 51). For example, current curve 49 starts to ramp down at a time 58 which precedes start of diastole 51 by a time t₂. Preferably, time 58 may be at a moment between about 50% to about 90% into the systolic phase. Thus, denoting the length of the systolic phase as tₛ, the ratio t₁/tₛ is preferably between 0.1 and 0.5.

As shown in FIG. 11, an average speed 59 is maintained as the instantaneous speed varies between elevated speed 53 and reduced speed 54. Average speed 59 may be determined in a conventional manner according to the physiological state of the patient. Offsets from average speed 59 for elevated speed 53 and reduced speed 54 may be substantially constant or may also be determined according to the physiological state of the patient.

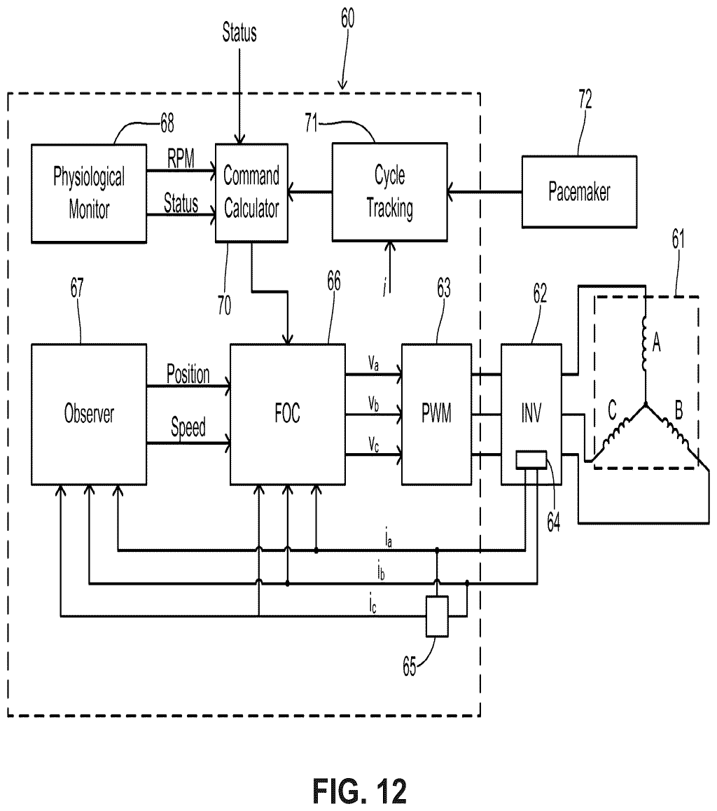

A pump system of the present invention is shown in greater detail in FIG. 12 wherein a controller 60 uses field oriented control (FOC) to supply a multiphase voltage signal to the pump motor which comprises a stator assembly 61 shown as a three-phase stator. Individual phases A, B, and C are driven by an H-bridge inverter 62 functioning as a commutation circuit driven by a pulse width modulator (PWM) circuit 63 in controller 60. A current sensing circuit 64 associated with inverter 62 measures instantaneous phase current in at least two phases providing current signals designated iₐ and i_{b}. A current calculating block 65 receives the two measured currents and calculates a current i_{c} corresponding to the third phase as known in the art. The measured currents are input to an FOC block 66 and to a current observer block 67 which estimates the position and speed of the impeller as known in the art. The impeller position and speed are input to FOC block 66.

An average target speed or rpm for operating the pump is provided by a physiological monitor 68 to FOC block 66. The average rpm may be set by a medical caregiver or may be determined according to an algorithm based on various patient parameters such heart beat. Monitor 68 may also generate a status signal for identifying whether the ventricle is in the initial, highly weakened state or whether a predetermined recovery has been obtained in the strength of the ventricle. The average rpm and the status signal are provided to a speed command calculator 70. The status signal can be used to determine whether or not the variable speed control of the invention should be used to unload the ventricle. The status signal can alternatively be externally provided to calculator 70 (e.g., by a physician via an HMI).

Command calculator 70 is coupled to a cycle tracking block 71 which maintains timing for a cardiac cycle reference. A current signal (e.g., currents iₐ, i_{b}, and i_{c} can be used in order to detect the cardiac cycle from the instantaneous blood flow, for example. More specifically, the controller may identify the heart rate by measuring time between current peaks in the speed control mode. Then the speed decrease can start at a calculated time after the occurrence of a current peak. The speed increase can start at a calculated time after the current minimum value is detected. This calculated time typically depends on the heart rate.

Alternatively, cycle tracking block 71 can be coupled to a pacemaker 72 in the event that the patient is using such a device. Conventional pacemakers have been constructed to continuously generate radio signals that contain information about pulse timing and other data. These sine-wave modulated signals can be received by a special receiver (not shown), where the signals are demodulated, digitized (if necessary), and transferred to cycle tracking block 71. Besides being located near the implanted pacemaker and connected by a cable or wirelessly to the controller (e.g., via BlueTooth), a receiver could be integrated with the controller or the pumping unit.

Based on the reference cycle timing from block 71, command calculator 70 determines an instantaneous speed (or magnitude of the current vector) to be used by FOC block 66. FOC block 66 generates commanded voltage output values Vₐ, V_{b}, and V_{c} which are input to PWM block 63. The Vₐ, V_{b}, and V_{c} commands may also be coupled to observer 67 for use in detecting speed and position (not shown). Thus, the speed is controlled to follow the curves shown in FIG. 10 and FIG. 11.

In one embodiment, the timing of the speed increases and decreases are determined as follows. At a constant pacing rate (i.e., constant beat rate), the time for starting the speed acceleration (e.g., at time 56 in FIG. 10) is:

$${t}_{acc} ( n + 1 ) = {t}_{p} ( n ) + 60 / N - {t}_{1} .$$

where tₚ(n) is the time of occurrence of a pacemaker pulse time signaling the start of the current cardiac cycle; N is the heart (pulse) rate in beat/min set by a pacemaker; and t_{acc}(n+1) is the time to increase the pump speed for the next cardiac cycle.

Similarly, the time to start deceleration (e.g., at a time 58 in FIG. 10) is:

$${t}_{decel} ( n + 1 ) = {t}_{a} ( n + 1 ) + {t}_{s}$$

where tₛ is the duration of systole. Systole typically lasts 30% to 50% of the cardiac cycle 60/N, and within a certain heart rate range it is fairly independent of the heart rate N. For example, for a heart rate N between 60-120 beats/min, tₛ is between 0.30 seconds and 0.25 seconds.

In an alternative embodiment, command calculator 70 and FOC block 66 are configured to operate the motor in a constant current mode (i.e., a constant torque mode). In this mode, the speed changes inversely with the pump load (i.e., the flow rate). Thus, an average speed is determined by the physiological monitor. The motor controller adjusts the current to obtain the desired average speed and to keep the current substantially constant. By keeping a constant current in the face of a load which varies within the cardiac cycle, the impeller speed automatically changes. FIG. 13 shows a load curve 75, wherein the load (i.e., flow rate) is high at 76 during systole and low at 77 during diastole. The load ramps up at 78 before the beginning of systole due to an increase of pressure within the ventricle and a decrease of pressure at the pump outlet (e.g., at the aorta). The load ramps down at 80 during the beginning of diastole.

In the current control mode, the pump flow increases (load increases) in the beginning of systole (at 78) and the speed curve 81 drops to a reduced speed 83. At the end of systole, the flow drops (at 80) and speed increases to an elevated speed 82. Thus, the speed increases and stays relatively high during diastole to help unload the ventricle by pumping out blood at the time it fills the ventricle. This is a natural behavior of the pump in the current control mode.

Either the variable speed control mode using a variable target speed or using the constant current approach of the invention can be combined with the conventional constant speed mode in order to adapt pump performance to the strength level of the patient's ventricle. In particular, the selection between the variable speed mode and the constant speed mode can be determined according to a physiologic capability of the patient. For example, the pump is set to operate in the constant current mode immediately following the implantation when the left ventricle is weak, thereby providing a greater level of ventricle unloading. With the patient's recovery, the pump may be set to operate in the constant speed mode, promoting higher flow pulsatility and a more natural physiologic response to the patient's activities.

The invention has now been described in detail for the purposes of clarity and understanding. However, it will be appreciated that certain changes and modifications may be practiced within the scope of the disclosure.

What is claimed is:

1. A circulatory assist system comprising:
a centrifugal ventricular assist device configured to pump blood from a ventricle of a patient to an artery of the patient, wherein the centrifugal ventricular assist device comprises a housing, a stator assembly, and a blood flow impeller, wherein the housing defines a blood flow inlet and a blood flow outlet, wherein the stator assembly comprises stator coils, wherein the blood flow impeller comprises a first disk portion, a second disk portion, and vanes extending between and separating the first disk portion and the second disk portion, wherein each of the first disk portion and the second disk portion comprises embedded magnetic segments for rotation and levitation of the blood flow impeller, and wherein the first disk portion defines a first disk portion central aperture configured for transit of a blood flow received through the blood flow inlet into the blood flow impeller for impelling radially outwardly between the first disk portion and the second disk portion via the vanes for output from the blood flow outlet;
a motor control circuit operable to supply drive currents to the stator coils;
a controller configured for controlling operation of the motor control circuit;
a sensing circuit configured to generate a stator drive signal indicative of a magnitude of the drive currents supplied to the stator coils by the motor control circuit or a magnitude of a drive power supplied to the stator coils via the drive currents; and
a blood flow cannula configured for connecting the blood flow outlet with the artery.

2. The circulatory assist system of claim 1, wherein:
the housing defines a central hub;
the second disk portion defines a second disk portion central aperture; and
the central hub extends at least partially through the second disk portion central aperture.

3. The circulatory assist system of claim 1, wherein:
the blood flow impeller has a rotation axis; and
the blood flow impeller is magnetically suspended parallel to the rotation axis during operation of the centrifugal ventricular assist device via the embedded magnetic segments.

4. The circulatory assist system of claim 1, wherein the housing defines a volute region that extends circumferentially and is configured to receive the blood flow from the blood flow impeller and redirect the blood flow to the blood flow outlet.

5. The circulatory assist system of claim 1, further comprising a battery pack for supplying electrical power for operation of the centrifugal ventricular assist device.

6. The circulatory assist system of claim 1, wherein the controller is configured to:
process the stator drive signal to determine a speed synchronization start point at which time a rotation rate of the blood flow impeller will begin a predetermined rotation rate variation that is synchronized with a patient's cardiac cycle; and
control the motor control circuit to modulate the rotation rate of the blood flow impeller to implement the predetermined rotation rate variation.

7. The circulatory assist system of claim 6, wherein:
the controller is configured to process the stator drive signal to determine an estimated current cardiac cycle pulse period for the patient; and
the controller is configured to determine the speed synchronization start point based on the estimated current cardiac cycle pulse period for the patient.

8. The circulatory assist system of claim 7, wherein the controller is configured to:
process the stator drive signal to determine at least two prior cardiac cycle pulse periods for the patient; and
determine the estimated current cardiac cycle pulse period for the patient based on the at least two prior cardiac cycle pulse periods.

9. The circulatory assist system of claim 6, wherein the stator drive signal is indicative of the magnitude of the drive currents supplied to the stator coils by the motor control circuit.

10. The circulatory assist system of claim 6, wherein the stator drive signal is indicative of the magnitude of the drive power supplied to the stator coils via the drive currents.

11. The circulatory assist system of claim 6, wherein the controller is configured to filter the stator drive signal to remove frequencies above 5 Hz.

12. The circulatory assist system of claim 6, wherein the controller is configured to process the stator drive signal to identify cardiac cycle features of the patient.

13. The circulatory assist system of claim 12, wherein the predetermined rotation rate variation begins prior to a systolic phase for the ventricle and ends before completion of the systolic phase for the ventricle.

14. The circulatory assist system of claim 13, wherein the controller is configured to control the motor control circuit to rotate the blood flow impeller at a constant rotational rate between implementations of the predetermined rotation rate variation.

15. The circulatory assist system of claim 14, wherein the rotation rate of the blood flow impeller during the predetermined rotation rate variation is higher than the constant rotation rate.

16. The circulatory assist system of claim 6, wherein the predetermined rotation rate variation is specified by a clinician.

17. A method of pumping blood from a ventricle of a patient to an artery of a patient, the method comprising:
operating a motor drive circuit of a centrifugal ventricular assist device to supply drive currents to stator coils of a motor stator to generate a magnetic field to drive rotation of a blood flow impeller that comprises a first disk portion, a second disk portion, and vanes extending between and separating the first disk portion and the second disk portion, wherein each of the first disk portion and the second disk portion comprises embedded magnetic segments for rotation and levitation of the blood flow impeller, and wherein the first disk portion defines a first disk portion central aperture configured for transit of a blood flow received through a blood flow inlet into the blood flow impeller for impelling radially outwardly between the first disk portion and the second disk portion via the vanes for output from a blood flow outlet; and
generating a stator drive signal indicative of a magnitude of the drive currents supplied to the stator coils by the motor drive circuit or a magnitude of a drive power supplied to the stator coils via the drive currents.

18. The method of claim 17, wherein:
the centrifugal ventricular assist device comprises a housing that defines a central hub;
the second disk portion defines a second disk portion central aperture; and
the central hub extends at least partially through the second disk portion central aperture.

19. The method of claim 18, wherein the housing defines a volute region that extends circumferentially and is configured to receive the blood flow from the blood flow impeller and redirect the blood flow to the blood flow outlet.

20. The method of claim 17, wherein:
the blood flow impeller has a rotation axis; and
the blood flow impeller is magnetically suspended parallel to the rotation axis during operation of the centrifugal ventricular assist device via the embedded magnetic segments.
